(19) United States
(12) Patent Application Publication
(10) Pub. No.: US 2005/0222518 A1
(43) Pub. Date: Oct. 6, 2005

(54) Biopsy and injection catheters

(75) Inventor: Nabil Dib, Phoenix, AZ (US)

Correspondence Address: Squire, Sanders & Dempsey L.L.P.;Two Renaissance Square, Suite 2700, 40 North Central Avenue, Phoenix, AZ, 85004-4498, US

(73) Assignee: GenoCell, LLC

(21) Appl. No.: 10/820,183

(22) Filed: Apr. 6, 2004

(51) Int. Cl. A61B 10/00

(52) U.S. Cl. 600/562

(57) ABSTRACT

Medical devices are delineated that may include a needle, a catheter and a sheath. Such devices may be used to take a biopsy from a patient and/or inject fluid into a patient, particularly in the patient's heart tissue. One or more ports may be located between the ends of the needle to facilitate the desired medical procedure. A relative motion restrictor may be included to restrict relative movement during needle deployment between the catheter and the sheath. Additionally, a driver may be employed to deploy the needle with one or more of a predefined force, acceleration and velocity to ease needle penetration.

### DESCRIPTION OF THE INVENTION

1. Field of the Invention

The present invention relates to medical devices, and, more particularly, to a medical device for taking a biopsy from a patient and/or injecting a fluid into a patient.

2. Background of the Invention

Catheters are widely used today in variety of medical procedures, such as for medical treatment of the heart. U.S. Pat. No. 6,689,103, which is incorporated herein by reference, discloses an exemplary catheter.

FIG. 1 represents a well-known technique to access the heart 16 of a patient 12 with a catheter 10. Specifically, catheter 10 is typically inserted through an incision into a patient's artery 14 and fed into heart 16 for treatment, which may include, among other things, taking a biopsy of heart tissue for analysis and/or injecting fluid into heart tissue.

FIGS. 2A-2C illustrate a problem commonly encountered with such treatments. FIG. 2A shows a working end of catheter 10 in contact with heart tissue of heart 16. FIGS. 2B and 2C show a needle 18 being deployed from catheter 10 for insertion into the heart tissue. Oftentimes, and undesirably, the resistance of the heart tissue forces catheter 10 away from the heart tissue as needle 18 is deployed, as represented by the arrows in FIGS. 2B and 2C, and prevents insertion of needle 18 into the heart tissue.

Exacerbating this problem are the relative ease by which catheter 10 may slide along needle 18, the flexibility of catheter 10 and the roughened texture and increased density of damaged heart tissue, as may result from fibrosis, including the formation of scar tissue following a heart attack. The structure of catheters for obtaining heart-tissue biopsies also contributes to the problem. For capturing a biopsy sample, such catheters 10 typically have a dull needle tip and/or some other structure that is inherently resistive, such as an abrasive surface, a pincher or the like, however, by having such structures located on the tip of needle 18, resistance often is elevated to the point of inhibiting smooth needle entry into the heart tissue. Also contributing to the problem is the current manner in which needle 18 is deployed, typically by a mechanism, such as a plunger external to patient 12, which may be moved by manual force, a solenoid, hydraulic pressure or pneumatic pressure. However deployed, needle 18 is generally moved too slowly and with insufficient force to ease insertion into the heart tissue.

The current inability to consistently and easily insert a needle from a catheter into a patient's tissue, such as heart tissue, limits the effectiveness of the desired medical treatment. Therefore, there is a need for catheters which overcome these and other problems of the prior art.

### SUMMARY OF THE INVENTION

In accordance with an embodiment of the invention, a medical device for taking a biopsy of material is disclosed, comprising a catheter having a catheter lumen with a proximal end and a distal end, and a needle for selectively moving to extend from the distal end of the catheter lumen to a first position to take the biopsy and to a second position within the catheter lumen. The needle may include a first port between the distal end of the needle and the proximal end of the needle for selectively opening and closing to take the biopsy of the material.

In accordance with another embodiment of the invention, a medical device for injecting a fluid into a material is disclosed, comprising a catheter having a catheter lumen with a proximal end and a distal end, and a needle for selectively moving to extend from the distal end of the catheter lumen to a first position to inject the fluid into the material and to a second position within the catheter lumen. The needle may include a first port between the distal end of the needle and the proximal end of the needle for selectively opening to inject the fluid into the material and closing.

In accordance with a further embodiment of the invention, a medical device for taking a biopsy of material is disclosed, comprising a catheter having a catheter lumen with a proximal end and a distal end, a needle for selectively moving to extend from the distal end of the catheter lumen to a first position to take the biopsy and to a second position within the catheter lumen, a sheath having a sheath lumen through which the catheter is selectively moved, and a driver for selectively moving the needle with one or more of a predefined force, a predefined acceleration and a predefined velocity to penetrate the material.

In accordance with a still another embodiment of the invention, a medical device for injecting a fluid into a material is disclosed, comprising a catheter having a catheter lumen with a proximal end and a distal end, a needle for selectively moving to extend from the distal end of the catheter lumen to a first position to inject the fluid into the material and to a second position within the catheter lumen, a sheath having a sheath lumen through which the catheter is selectively moved, and a driver for selectively moving the needle with one or more of a predefined force, a predefined acceleration and a predefined velocity to penetrate the material.

Additional objects and advantages of the invention will be set forth in part in the description which follows, and in part will be obvious from the description, or may be learned by practice of the invention. The objects and advantages of the invention will be realized and attained by means of the elements and combinations particularly pointed out in the appended claims.

It is to be understood that both the foregoing general description and the following detailed description are exemplary and explanatory only and are not restrictive of the invention, as claimed.

The accompanying drawings, which are incorporated in and constitute a part of this specification, illustrate several embodiments of the invention and together with the description, serve to explain the principles of the invention.

### BRIEF DESCRIPTION OF THE DRAWINGS

FIG. 1 illustrates a well-known technique to access the heart of a patient with a catheter.

FIGS. 2A-2C illustrate a problem commonly encountered when employing a catheter to take a biopsy of tissue and/or to inject fluid into tissue.

FIG. 3 is a simplified block diagram of a system including a medical device for taking a biopsy of tissue and/or injecting fluid into tissue, in accordance with systems and methods consistent with the present invention.

FIG. 4 is a simplified flowchart illustrating a method of employing a medical device for taking a biopsy of tissue and/or injecting fluid into tissue, in accordance with systems and methods consistent with the present invention.

FIGS. 5A-5E show cross-sectional views of a working end of a medical device for taking a biopsy of tissue and/or injecting fluid into tissue, in accordance with systems and methods consistent with the present invention.

FIG. 6 shows a planar view of the front of the working end of a medical device for taking a biopsy of tissue and/or injecting fluid into tissue, in accordance with systems and methods consistent with the present invention.

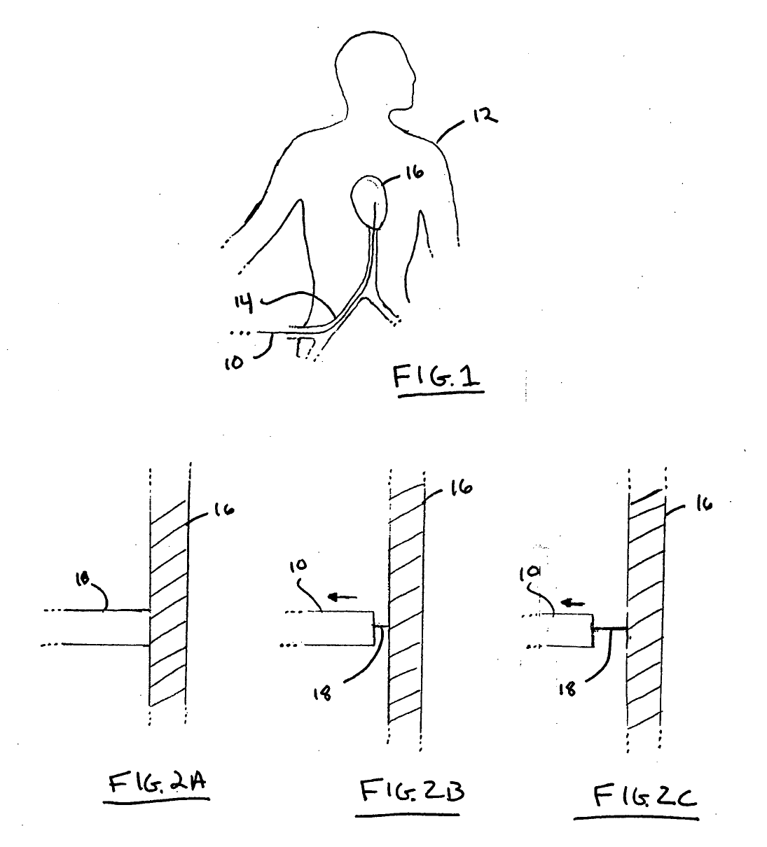

### DESCRIPTION OF THE EMBODIMENTS

Reference will now be made in detail to the present exemplary embodiments of the invention, examples of which are illustrated in the accompanying drawings. Wherever possible, the same reference numbers will be used throughout the drawings to refer to the same or like parts.

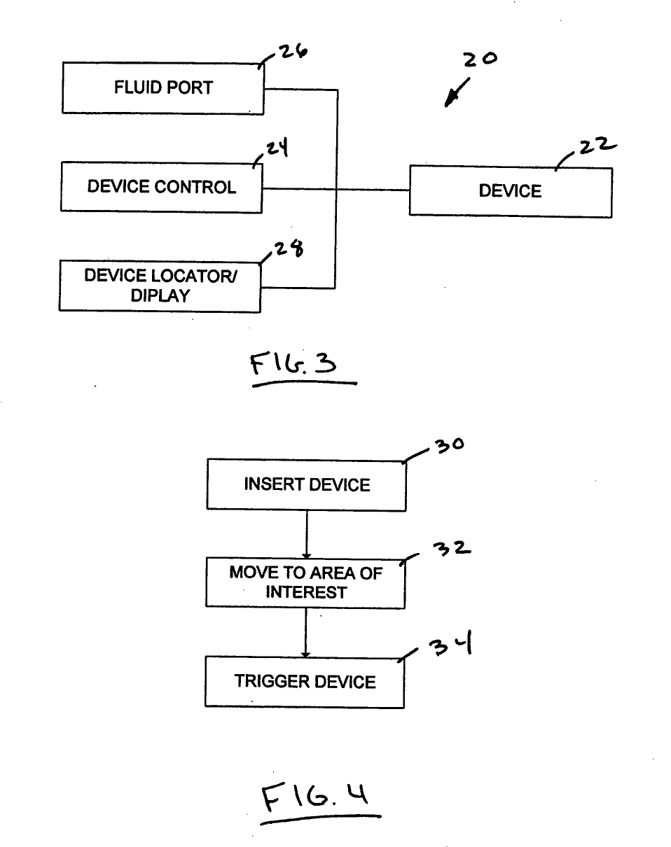

Referring to FIG. 3, a medical system 20 may be utilized for performing any desired medical procedure. In an exemplary embodiment, medical system 20 may be employed to take a biopsy of and/or inject a fluid into a tissue, such as heart tissue. Medical system 20 may include one or more medical devices 22 (hereafter “medical device 22”), one or more medical device control systems 24 (hereafter “control system 24”), one or more fluid ports 26 (hereafter “port 26”) and one or more medical device locator and display systems 28 (hereafter “display system 28”).

Medical device 22 may comprise any structure suitable for performing any desired medical procedure, e.g., taking a biopsy and/or injecting a fluid into a tissue. An injected fluid may entrain any material for the desired procedure, e.g., cells or genes. Moreover, medical device 22 may comprise one device for performing multiple procedures or separate devices, each such separate device for performing one or several desired procedures.

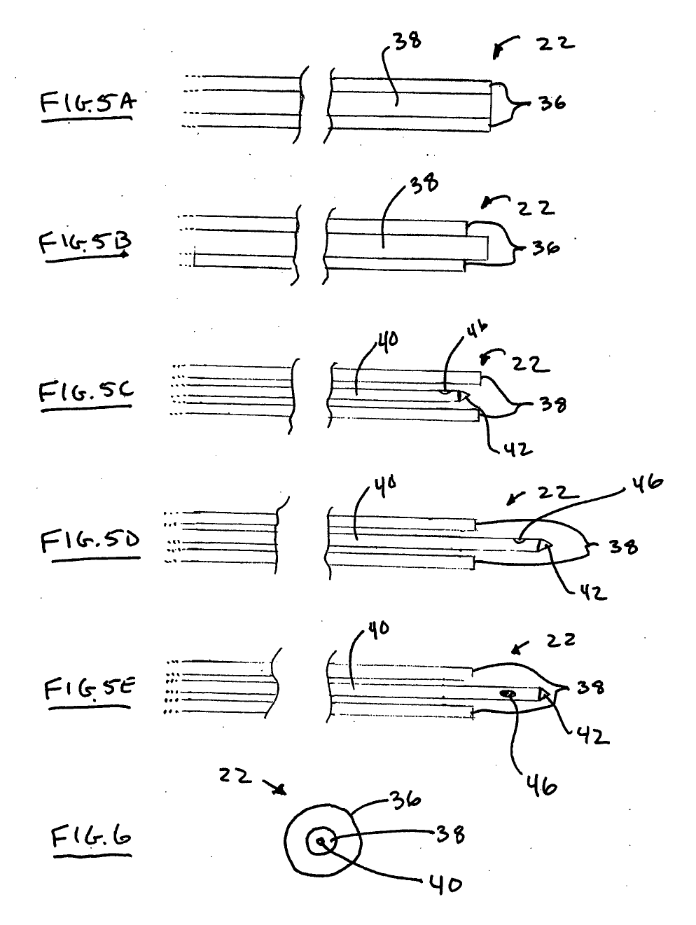

In one embodiment, medical device 22 may comprise a catheter 38 and a needle 40, as represented in FIGS. 5A-5E in which needle 40 is not shown in FIGS. 5A and 5B for purposes of drawing simplification. Catheter 38 may comprise any catheter of material and size suitable for purposes of the desired medical procedure and typically extends from a working end, the right end shown in FIGS. 5A-5E, through patient 12, to a medical professional performing the desired medical procedure. Needle 40 may comprise any needle of material and size suitable for purposes of the desired medical procedure and typically resides within a lumen of catheter 38. A needle guide, such as a guide wire (not shown), is typically connected to the non-working end of needle 40 and extends through the catheter lumen to a medical professional performing the desired medical procedure.

In another embodiment, medical device 22 may comprise catheter 38, needle 40 and a sheath 36, as represented in FIGS. 5A-5E in which needle 40 is not shown in FIGS. 5A and 5B and sheath 36 is not shown in FIGS. 5C-5E for purposes of drawing simplification. Catheter 38 and needle 40 may comprise structures similar to those described with respect to the preceding embodiment of medical device 22. Sheath 36 may comprise any sheath of material and size suitable for purposes of the desired medical procedure and typically extends from a working end, the right end shown in FIGS. 5A-5E, through patient 12, to a medical professional performing the desired medical procedure.

To permit loading fluid into medical system 20 for injection into patient 12, medical system 20 may include port 26. Port 26 is typically located outside of patient 12, accessible to a medical professional performing the desired medical procedure and in fluid communication with one or more delivery ports in needle 40. The delivery port or ports may be located anywhere on needle 40, including, as shown in FIGS. 5C-5E, one or more delivery ports 46 (hereafter “port 46”) located between the ends of needle 40. Thus, fluid loaded into port 26 may be injected into patient 12 at port 46. Similarly, medical system 20 may be employed to take a biopsy of material, such as tissue in patient 12, at port 46 for removal through port 26.

Any control system 24 may be employed that is suitable for controlling medical device 22, port 26 and/or display system 28, as desired for the selected medical procedure. Control system 24 may employ manual control and/or automatic control. Manual control typically employs a user to control, e.g., move, an object, either directly, i.e., the user touches the object to control it, or indirectly, i.e., the user touches an intermediary structure to control the desired object. Automatic control typically employs one or more programs which, when executed, perform programmed operations to control, e.g., move, the desired object. Whether manual control and/or automatic control is employed to control medical device 22, port 26 and/or display system 28 for the selected medical procedure, any power source, such as manual, electric, electromechanical, hydraulic, pneumatic and the like, may be utilized to supply force to control, e.g., move the selected object as desired.

To control sheath 36, any sheath controller and/or sheath-control technique may be used to insert, move and/or in any way employ sheath 36 to perform the desired medical procedure. To control catheter 38, any catheter controller and/or catheter-control technique may be used to insert, move and/or in any way employ catheter 38 to perform the desired medical procedure. FIGS. 5A and 5B together illustrate an exemplary controlled movement of sheath 36 and/or catheter 38. More specifically, FIG. 5A shows coplanar alignment of the working ends of sheath 36 and catheter 38. FIG. 5B shows a changed alignment, which may result from movement of sheath 36 and/or catheter 38. For example, a medical professional rendering the desired medical procedure may, by moving sheath 36 and/or catheter 38, vary the “unsheathed” length, if any, of catheter 38 that may extend outside of sheath 36. Reducing the “unsheathed” length of catheter 38, i.e., covering more of catheter 38 with sheath 36, may tend to constrain or limit any potential bowing of catheter 38 that, in the absence of sheath 36, may otherwise occur during needle deployment toward an intended target of tissue. Reducing catheter bowing may tend to improve the ease with which needle 40 may enter tissue.

Control system 24 may also selectively restrict relative movement between sheath 36 and catheter 38, which may include the sliding of catheter 38 within the lumen of sheath 36. Any structure that is suitable for this purpose may be employed. For example, a mechanical stop or an electromechanical stop (hereafter inclusively “stop”) may be used to selectively restrict relative movement between sheath 36 and catheter 38, e.g., restricting any movement, such as the sliding of catheter 38 within the lumen of sheath 36.

A stop may comprise a mechanical structure for, at a selected time, restricting the relative movement between sheath 36 and catheter 38, e.g., a clamp that may be selectively engaged to hold together a portion of sheath 36 and a portion of catheter 38 with some predefined force. A stop may also comprise an electromechanical structure for, at a selected time, restricting the relative movement between sheath 36 and catheter 38, e.g., one or more selectively-expandable regions on sheath 36 and/or catheter 38 that may be expanded to restrict relative motion between sheath 36 and catheter 38 during, for example, needle deployment toward an intended target of tissue. Control of a stop, whatever its structure, may be manual and/or automatic. Considering automatic control of a stop, for example, at a predetermined time, e.g., before or during needle deployment toward an intended target of tissue, the stop may automatically activate to restrict the relative movement between sheath 36 and catheter 38 for a predetermined period of time during needle deployment and then release the applied restriction against relative movement between sheath 36 and catheter 38 at a predetermined time, e.g., after needle 40 returns to within catheter 38.

Increased resistance to relative movement between sheath 36 and catheter 38, e.g., the sliding of catheter 38 within the lumen of sheath 36, may tend to prevent undesirable movement of catheter 38 away from target tissue that, in the absence of such resistance, may otherwise occur during needle deployment toward an intended target of tissue. Limiting movement of catheter 38 away from target tissue during needle deployment may tend to improve the ease with which needle 40 may enter tissue.

Needle 40 may also be controlled manually and/or automatically. Regardless of the manner of control, needle 40 may be driven to provide one or more different movements to perform the desired medical procedure. Such movements may include: 1) movement of needle 40 within the lumen of catheter 38, without extending needle 40 outside of the working end of catheter 38; 2) deploying needle 40, i.e., moving a portion of needle 40 to extend outside of the working end of catheter 38; 3) returning needle 40 within the lumen of catheter 38; 4) rotating needle 40 along its axis; 5) opening port 46; 6) closing port 46; and 7) any other desired movement.

In the case of manual needle control, for example, a medical professional rendering the desired medical procedure may depress a needle actuator, which may initiate and apply a manual force or other type of force to produce a desired needle movement, e.g., deploying needle 40. An opposing force may be provided by, for example, an opposing spring-loaded mechanism in the needle actuator, which may return needle 40 back within catheter 38 when the needle actuator is released. In the case of automatic needle control, for example, a medical professional rendering the desired medical procedure may position the medical device 22 in a position that automatically triggers a predefined operation of needle 40. Alternatively, once the medical professional confirms that medical device 22 is in the desired position, using, for example, display system 28, the medical professional may depress a needle actuator, causing needle 40 to perform a predefined operation.

By way of example, a predefined needle operation may include: 1) deploying needle 40 to enter tissue; 2) opening port 46, either before or after needle 40 enters the tissue to, for example, permit injecting a fluid or taking a biopsy; 3) rotating needle 40 along its axis, either before or after needle 40 enters the tissue to, for example, facilitate taking a biopsy; 4) returning needle 40 within the lumen of catheter 38; and 5) closing port 46 while needle 40 is within tissue to, for example, facilitate taking a biopsy, or after needle 40 is removed from the tissue. When deploying needle 40, a driver may be employed to selectively move needle 40 with a predefined force, a predefined acceleration, and/or a predefined velocity, to improve the ability of needle 40 to easily penetrate the tissue.

Display system 28 may locate and display medical device 22 within patient 12. If automatic triggering is used based on the location of medical device 22, display system 28 may provide such location information. Display system 28 may employ any structure and/or method suitable for such purposes. A sensor 42 may be located on medical device 22, e.g., on needle 40, and in communication with display system 28 to provide location information for medical device 22 back to display system 28. Sensor 42 may comprise any sensor that is suitable for this purpose and may be compatible with electrical and/or magnetic fields.

FIG. 4 illustrates a method of employing medical device 22 for taking a biopsy of tissue and/or injecting fluid into tissue. At step 30, a medical professional may insert medical device 22 using any technique. At step 32, the medical professional may move medical device 22 to an area of interest using any technique and may employ display system 28 to monitor this process. At step 34, medical device 22 may be triggered to perform the desired medical operation, such as injecting a fluid into or taking a biopsy from tissue. Triggering may be automatic or manually selected.

FIGS. 5A-5B together illustrate an exemplary controlled movement of sheath 36 and/or catheter 38. More specifically, FIG. 5A shows coplanar alignment of the working ends of sheath 36 and catheter 38. FIG. 5B shows a changed alignment, which may result from movement of sheath 36 and/or catheter 38. Needle 40 is not shown in FIGS. 5A and 5B for purposes of drawing simplification.

Referring to FIGS. 5C-5E, sheath 36 is not shown for purposes of drawing simplification. FIG. 5C shows needle 40 within a lumen of catheter 38. FIG. 5D shows needle 40 being deployed from catheter 38, as represented by the arrow pointing away from the working end of catheter 38. As shown in FIG. 5D, port 46 may be closed during needle deployment, however, port 46 may be opened during needle deployment or at any time suitable for the selected medical procedure. FIG. 5E shows needle 40 being rotated along its axis and port 46 in an open position to facilitate, for example, the taking of a biopsy. At a suitable time, needle 40 may be returned within the lumen of catheter 38, as represented by FIG. 5C.

FIG. 6 is a planar view from the front of the working end of medical device 22. As shown, sheath 36, catheter 38 and needle are coaxial, however, they need not be aligned.

Other embodiments of the invention will be apparent to those skilled in the art from consideration of the specification and practice of the invention disclosed herein. It is intended that the specification and examples be considered as exemplary only, with a true scope and spirit of the invention being indicated by the following claims.

1. A medical device for taking a biopsy of material, comprising:
a catheter having a catheter lumen with a proximal end and a distal end; and
a needle for selectively moving to extend from the distal end of the catheter lumen to a first position to take the biopsy and to a second position within the catheter lumen, said needle having a first port between the distal end of the needle and the proximal end of the needle for selectively opening and closing to take the biopsy of the material.

2. The medical device of claim 1 further including a sheath having a sheath lumen through which the catheter is selectively moved.

3. The medical device of claim 2 further including means for selectively restricting relative movement between the sheath and the catheter.

4. The medical device of claim 1 further including a driver for selectively moving the needle with one or more of a predefined force, a predefined acceleration and a predefined velocity to penetrate the material.

5. The medical device of claim 4 wherein the driver selectively rotates the needle.

6. The medical device of claim 1 further including a sensor on the device for providing signals for determining position of the device.

7. The medical device of claim 6 wherein the sensor is on the needle.

8. The medical device of claim 1 further including a second port in fluid communication with the first port to retrieve the biopsy material from the first port.

9. A medical device for injecting a fluid into a material, comprising:
a catheter having a catheter lumen with a proximal end and a distal end; and
a needle for selectively moving to extend from the distal end of the catheter lumen to a first position to inject the fluid into the material and to a second position within the catheter lumen, said needle having a first port between the distal end of the needle and the proximal end of the needle for selectively opening to inject the fluid into the material and closing.

10. The medical device of claim 9 further including a sheath having a sheath lumen through which the catheter is selectively moved.

11. The medical device of claim 10 further including means for selectively restricting relative movement between the sheath and the catheter.

12. The medical device of claim 9 further including a driver for selectively moving the needle with one or more of a predefined force, a predefined acceleration and a predefined velocity to penetrate the material.

13. The medical device of claim 12 wherein the driver selectively rotates the needle.

14. The medical device of claim 9 further including a sensor on the device for providing signals for determining position of the device.

15. The medical device of claim 14 wherein the sensor is on the needle.

16. The medical device of claim 9 further including a second port in fluid communication with the first port and a supply of the fluid.

17. A medical device for taking a biopsy of material, comprising:
a catheter having a catheter lumen with a proximal end and a distal end;
a needle for selectively moving to extend from the distal end of the catheter lumen to a first position to take the biopsy and to a second position within the catheter lumen;
a sheath having a sheath lumen through which the catheter is selectively moved; and
a driver for selectively moving the needle with one or more of a predefined force, a predefined acceleration and a predefined velocity to penetrate the material.

18. The medical device of claim 17 further including means for selectively restricting relative movement between the sheath and the catheter.

19. A medical device for injecting a fluid into a material, comprising:
a catheter having a catheter lumen with a proximal end and a distal end;
a needle for selectively moving to extend from the distal end of the catheter lumen to a first position to inject the fluid into the material and to a second position within the catheter lumen;
a sheath having a sheath lumen through which the catheter is selectively moved; and
a driver for selectively moving the needle with one or more of a predefined force, a predefined acceleration and a predefined velocity to penetrate the material.

20. The medical device of claim 19 further including means for selectively restricting relative movement between the sheath and the catheter.
